(19) United States
(12) Patent Application Publication
(10) Pub. No.: US 2018/0042623 A1
(43) Pub. Date: Feb. 15, 2018

(54) Blood Clot Aspiration Catheter

(71) Applicant: Stanley Batiste, Granite Bay, CA (US)

(72) Inventor: Stanley Batiste, Granite Bay, CA (US)

(21) Appl. No.: 15/675,670

(22) Filed: Aug. 11, 2017

Related U.S. Application Data
(60) Provisional application No. 62/373,846, filed on Aug. 11, 2016.
(60) Provisional application No. 62/416,612, filed on Nov. 2, 2016.
(60) Provisional application No. 62/420,425, filed on Nov. 10, 2016.
(60) Provisional application No. 62/426,111, filed on Nov. 23, 2016.
(60) Provisional application No. 62/472,474, filed on Mar. 16, 2017.

(51) Int. Cl. A61B 17/22; A61M 39/24

(52) U.S. Cl. CPC A61B 17/22; A61M 39/24; A61M 2205/7545; A61B 2017/22038; A61B 2017/22067; A61B 2017/22079; A61M 2039/2426; A61B 2217/007

(57) ABSTRACT

A blood clot aspiration catheter includes an aspiration/flushing lumen having a distal portion that is insertable into a blood vessel of a patient, an aspiration device configured to create an aspiration phase to aspirate clot and blood into the aspiration/flushing lumen and a flushing phase to flush non-clotted blood from the aspiration/flushing lumen, a one-way valve configured to allow clot and blood to flow through the aspiration/flushing lumen during the aspiration phase and to block flow during the flushing phase, and filter valves configured to filter non-clotted blood to be flushed from the aspiration/flushing lumen.

### CROSS-REFERENCE TO RELATED APPLICATIONS

This application claims priority to U.S. Provisional Application No. 62/373,846 which was filed on Aug. 11, 2016; U.S. Provisional Application No. 62/416,612 which was filed on Nov. 2, 2016; U.S. Provisional Application No. 62/420,425 which was filed on Nov. 10, 2016; U.S. Provisional Application No. 62/426,111 which was filed on Nov. 23, 2016; and U.S. Provisional Application No. 62/472,474 which was filed on Mar. 16, 2017. The contents of each of the above applications are incorporated by reference.

### BACKGROUND

### 1. Field

The disclosed embodiments relate to thrombectomy catheters which are used in the human vascular system to aspirate blood clots.

### 2. Related Art

Deep vein thrombosis (DVT) is a common problem and causes significant morbidity and mortality in the United States and throughout the world. DVT is caused when a blood clot forms in the deep veins of the legs. These blood clots typically occur due to slow or reduced blood flow through the deep veins such as when the patient cannot ambulate or otherwise efficiently circulate their blood. Another cause of inefficient circulation may be due to structural damage to the veins such as general trauma or subsequent to surgical procedures. Additionally, a blood clot may form in a deep vein due to a particular medical condition or a propensity for the patient to have a hypercoagulability state. For example, a woman on birth control who smokes has an increased risk of forming blood clots and is thus predisposed to DVT.

The result and clinical significance of DVT is when the clot breaks free from its location in the deep vein of the leg, the clot travels through the circulatory system and may eventually lodge in a location that is adverse to the patient's health. For example, the clot may dislodge from a location in the deep vein of the patient's leg and migrate through the heart and come to rest in the patient's lung causing a pulmonary embolism (PE) resulting in restricted circulation and can cause sudden death for the patient.

DVT & PE are currently prevented in several ways including anticoagulation therapy, thrombectomy, thrombolysis, and inferior vena cava filter (IVC filter) placement. Anticoagulation therapy utilizes various medications that reduce the patient's propensity for forming blood clots.

Thrombolysis is a medical technique that is performed for treatment of a DVT, in which various medicines are infused into the region of the clot that subsequently causes the clot to dissolve. This form of treatment has the disadvantage that the medication may cause bleeding at other sites such as within the brain. For example, if a patient has previously had a minute non-clinical stroke, the medication used in a thrombolysis may cause a previously healed vessel to bleed within the patient's head.

Thrombectomy is a procedure generally performed for treatment of a DVT, in which a blood clot is extracted from the vein using a surgical procedure or by way of an intravenous catheter using a mechanical aspiration or extraction method. This form of treatment can be technically challenging because the catheter must be steered or navigated to a specific location in order to extract the clot. Currently, there are many different types of mechanical thrombectomy devices using several different means of clot removal. Extraction devices use expanding, removal stents and net/filter types of devices that trap clots which are then pulled out through the original vascular entry point. With aspiration devices, usually either a means of creating low pressure and suction through a catheter is used. A second type of aspiration catheter utilizes a high velocity jet directed back into the catheter to create low pressure and suction using the Bernoulli principle. Both aspiration methods can remove clots out of vessels but are limited because once most of the clot is removed, blood replaces the space resulting in the catheters aspirating mainly blood which makes it hard to get the remaining clot out. These challenges with blood loss during aspiration limits that amount of clot that can be removed and is the genesis of the new clot aspiration catheter in the embodiments described below.

### SUMMARY

Deep vein thrombosis (DVT) is a common problem and causes significant morbidity and mortality in the United States and throughout the world. DVT is caused when a blood clot forms in the deep veins of the legs. Currently therapy involves using pharmacological and/or mechanical means of removing the clot from the vessels. With mechanical thrombectomy, clot is removed by several means including aspiration of the clot via suction through a catheter.

Although this can be effective in clot removal, a large portion of the clot may be left behind due to the limitation of non-clotted blood aspiration. Once the clot is removed it is replaced within the vessel by non-clotted blood which are then aspirated together. With more chronic clot there is even greater limitation as the clot is adherent to the vessel wall, and therefore the procedure will take more time, generally yielding more non-clotted blood.

In the typical therapy environment of an angiography lab, the clot is not visible while the thrombectomy is being performed making it an even more difficult task. The ideal thrombectomy catheter for the best clot aspiration is one of a large caliber. However, large caliber catheters are not ideal once non-clotted blood has replaced the aspirated clot within the vein. This is because, as explained above, the large-caliber catheter will begin to aspirate a large amount of non-clotted blood.

There are systems that have addressed this by capturing the clot by means of filtration outside the body and then having a second venous access for blood return. While operable, this method requires that there is a second entry into an additional vessel and also requires, in most setting, that a blood perfusion specialist be involved. With these systems (AngioVac by AngioDynamics), clot is aspirated with large amounts of non-clotted blood, the aspirated clot and blood are filtered, and the non-clotted blood is returned to the body via a second access site. The systems that do not allow filtration and reinfusion of blood (Indigo by Penumbra) are limited because once significant non-clotted blood is removed from the patient, the therapy must be terminated for obvious health reasons.

The disclosed embodiments solve the noted issues by filtering the blood within the patient's body by means of a filtration catheter in a closed system using a cycle of positive and negative pressures. The designs allow an operator to ultimately acquire only clot from the vessel and retuning non-clotted blood, allowing long working times and complete clot removal. The embodiments include a set of unidirectional valves and filters that separate and direct clot and non-clotted blood.

Catheters according to a first embodiment are placed in a vessel like other catheters and may have similar proximal attachments like standard vascular catheters. Within the distal tip, there is an entry valve that, with suction applied to the proximal catheter lumen, will aspirate both clot and non-clotted blood into the catheter lumen. The catheter is then advanced by the operator deeper into the clot and aspiration is again applied acquiring both clot and non-clotted blood.

Once there is substantial blood and clot within the catheter, positive pressure is applied to the proximal end of the lumen increasing the pressure within the lumen. This increase pressure forces non-clotted blood through a series of micro-filter valves on the wall of the catheter. With constant positive pressure, the non-clotted blood will be separated and return to the patient's vein, and the clot will remain in the catheter. The negative pressure cycle then repeats with the operator moving the catheter deeper into the clot and again both clot and non-clotted blood are removed. The positive pressure cycle is then repeated filtering the non-clotted blood back into the body. These cycles are then repeated until there is complete clot removal. When finished, the catheter is then removed.

A second embodiment of the design employs a parallel flush catheter alongside of the aspiration catheter to allow the operator to apply a fluid flush that is under pressure creating retrograde flow in aspiration catheter clearing out the clot. This eliminates clot from the catheter without having to remove the catheter from its position in the vessel.

In another embodiment, a standard thrombectomy type balloon such as a Fogerty catheter is used remove the clot from the catheter. The balloon is advanced in an ante grade fashion through the catheter in an un-inflated state, and is then inflated and pulled back through the catheter dragging the clot in front of it.

The clot then can be collected in a holding container with can be mounted on the aspiration catheter. Clot can also be collected directly out of the proximal end of the catheter.

There are many methods to increase and decrease pressures in a catheter with the most common using a syringe. Numerous others including suction pumps and vacuum bottles with regulators may also be used.

A third embodiment of the aspiration catheter filters the blood outside of the body by keeping the clot inside the catheter and allowing non-clotted blood to pass through small filters. Instead of having the non-clotted blood return directly into the vessel when filtered outside the blood is first collected in an external cylinder surrounding the catheter with return tubing that leads to the distal aspect of the primary catheter. The captured clot then can be directed by way of valves into a holding container.

A forth embodiment of the design uses a parallel convergence of the catheters at the mid-point where there are microvalves to filter the clotted and non-clotted blood. The filtration is helped by a small resistance valve creating back-pressure to separate the blood elements. When the resistance is overcome by pressure the valve allows passage of the filtered clotted blood.

A fifth embodiment of the design employs the use of a filter mechanism directly within the syringe or vessel attached to the syringe to separate the blood clot from the normal, non-clotted blood. When the clot is aspirated into the syringe the valve opens allowing flow distal to the filter. Once forward pressure on the plunger of the syringe cause the forward flow to close the valve with then acts as the filter. The clot is filtered and then can be separately eliminated from the syringe by means of a second outflow attached to the syringe constricted more proximal within the syringe with respect to the filter valve.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

FIG. 1 is a segmented view of a blood clot aspiration catheter showing a proximal end and a distal end, according to an exemplary embodiment.

FIG. 2A shows the distal portion of the catheter of FIG. 1 with low pressure and flow toward the proximal end with the end valve open, and FIG. 2B shows the distal portion of the catheter of FIG. 1 with high pressure and flow directed toward the distal end with the end valve closed and flow through the micro-valves.

FIG. 3A shows embodiments of the end valve open, and FIG. 3B shows embodiments of the end valve closed.

FIG. 4A shows an enlarged view of a single micro-valve, and FIG. 4B shows a perimeter of the microvalve shown in FIG. 4A.

FIG. 5A, FIG. 5B, and FIG. 5C show views of the distal portion of the catheter with enlarged views of micro-valve function with various flow directions.

FIG. 6A shows an aspiration filter with micropores with the flow direction with aspiration, and FIG. 6B shows the flow direction with flushing with filtered fluid exiting the catheter through the micropores, according to an exemplary embodiment.

FIG. 7A shows an aspiration catheter pre-engaged with suction and proximal flow, FIG. 7B shows the catheter engaged in the clot with unit clot removal, FIG. 7C, FIG. 7D, and FIG. 7E show further clot removal, FIG. 7F shows reversal of flow with expulsion of non-clotted blood and compaction of the clot units, and FIG. 7G shows reversal of flow and clot aspiration through the distal end valve.

FIG. 8A shows a blood clot aspiration catheter with a small parallel catheter, according to an exemplary embodiment, FIG. 8B shows an enlarged distal portion of the catheter of FIG. 8A, FIG. 8C shows an end view of the catheter of FIG. 8A, FIG. 8D shows fluid pressure forced through the small parallel catheter, FIG. 8E shows an aspirated clot within the catheter of FIG. 8A, and FIG. 8F shows clot removal from the catheter.

FIG. 9 shows a blood clot aspiration catheter with multiple aspiration valves along the catheter length, according to one exemplary embodiment.

FIG. 10A shows a catheter with multiple microvalves along the catheter length and an accompanying catheter cover, according to one exemplary embodiment, and FIG. 10B shows the catheter of FIG. 10A with the cover attached.

FIG. 11A shows an aspiration catheter with a central aspiration lumen with numerous outflow valves into a return catheter channel which continues to an outflow lumen, according to an exemplary embodiment, and FIG. 11B shows an enlarged view of the distal end of the catheter shown in FIG. 11A.

FIG. 12A shows an aspiration catheter with a central aspiration lumen with numerous outflow valves into a return catheter channel which continues to the outflow lumen, according to an exemplary embodiment, and FIG. 12B shows an enlarged view of the distal end of the catheter shown in FIG. 12A.

FIG. 13A shows an enlarged view of a distal end of an aspiration catheter with an internal filtration catheter, FIG. 13B shows a distal portion of the aspiration catheter of FIG. 13A during an aspiration phase, FIG. 13C shows the aspiration catheter of FIG. 13A during a flushing phase, and FIG. 13D shows an enlarged view of the distal end of the aspiration catheter of FIG. 13A during the flushing phase, according to one exemplary embodiment.

FIG. 14 shows an aspiration catheter that employs a port with a valve that directs clot to a holding container, according to an exemplary embodiment.

FIG. 15A shows a distal end and an enlarged section view of an aspiration catheter with slits creating the micro-valves, according to one exemplary embodiment, FIG. 15B is an enlarged view of the slit in an open state, FIG. 15C shows an internal filter to be inserted within the aspiration catheter of FIG. 15A, and FIG. 15D shows an enlarged view of the slit in an open state with the internal filter.

FIG. 16 illustrates a graph showing a possible cycle of aspiration and flushing over time, according to an exemplary embodiment.

FIG. 17A shows a deflated balloon advanced through the clot, FIG. 17B show inflation of the balloon, and FIG. 17C shows the balloon pulled pack through the catheter to remove the clot.

FIG. 18A shows a motor driven cylinder creating an aspiration and a flush cycle, and FIG. 18B shows a hand operated syringe to create an aspiration and a flush cycle.

FIG. 19A shows a blood clot aspiration catheter facilitating blood filtration outside of a patient, according to an exemplary embodiment, FIG. 19B shows a clot aspirated into the filter of FIG. 19A, FIG. 19C shows a proximal portion of the catheter of FIG. 19A with the clot aspirated into the proximal portion, FIG. 19D shows positive pressure into the proximal portion of the catheter of FIG. 19A, and FIG. 19E shows clot collection and filtered blood return using the catheter shown in FIG. 19A.

FIG. 20A shows a removable aspiration filter for an aspiration catheter, according to one exemplary embodiment, and FIG. 20B shows the filter in a removed state.

FIG. 21A shows a removable aspiration filter with a blind end filtration unit, FIG. 21 B shows a blind end filtration unit with microvalves and a mesh filtration system, and FIG. 21C shows the filter in a removed state.

FIG. 22A shows another embodiment of a blood clot aspiration catheter, FIG. 22B shows an aspiration phase of the catheter in FIG. 22A, FIG. 22C shows a flush state of the catheter in FIG. 22A, and FIG. 22D shows a clot disposal phase of the catheter in FIG. 22A.

FIG. 23A shows another embodiment of a blood clot aspiration catheter with a parallel guidewire lumen, and FIG. 23B shows a guidewire inserted into the lumen.

FIG. 24A shows another embodiment of a blood clot aspiration catheter with a conjoined aspiration lumen and filtration lumen attached with a connection having micro filters, FIG. 24B shows aspiration using a syringe into the aspiration catheter of FIG. 24A, FIG. 24C shows further aspiration of a clot into a syringe, FIG. 24D shows forward fluid pressure from the syringe moving the clot into the filtration lumen with non-clotted blood forced through microvalves, and FIG. 24E shows opening of a flow valve and passage of clot into a holding container.

FIG. 25 shows another embodiment of a blood clot aspiration catheter with a valve and filter combined and residing within a syringe

FIG. 26A shows a combination filter/valve with a central axis rod holding valve components through attached hinges that have valve stops extending from the hinges, according to one exemplary embodiment, and FIG. 26B shows the combination filter/valve in an open position.

FIG. 27 shows the filter/valve motion within a syringe based on neutral conditions, syringe aspiration, and flushing.

FIG. 28A shows a blood clot aspiration catheter using the combination filter valve in an aspiration phase, and FIG. 28B shows the aspiration catheter in a flush stage.

The components in the figures are not necessarily to scale, emphasis instead being placed upon illustrating the principles of the invention. In the figures, like reference numerals designate corresponding parts throughout the different views.

### DETAILED DESCRIPTION OF EMBODIMENTS

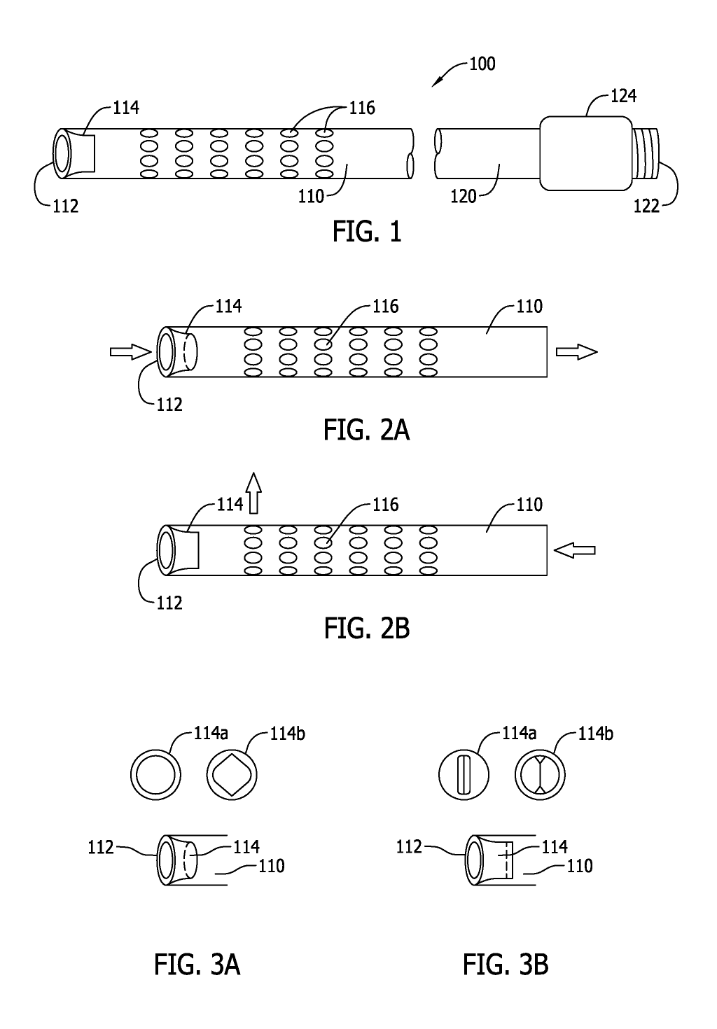

FIG. 1 is a segmented view of a blood clot aspiration catheter showing a proximal end and a distal end, according to an exemplary embodiment. A blood clot aspiration catheter 100 comprises a distal portion 110 and a proximal portion 120. The length extending from the distal portion 110 to the proximal portion 120 is not shown in FIG. 1, but is of sufficient length to access a blood vessel of a patient. The distal portion 110 comprises a distal end 112 to which a one-way valve 114 is attached. A plurality of micro-valves 116 are disposed on distal portion 110 of the catheter 100. The proximal portion 120 may comprise, for example a luer lock hub 122 and locking device 124 as is known in the art.

FIG. 2A shows the distal portion of the catheter of FIG. 1 with low pressure and flow toward the proximal end with the end valve open, and FIG. 2B shows the distal portion of the catheter of FIG. 1 with high pressure and flow directed toward the distal end with the end valve closed and flow through the micro-valves. To aspirate a clot within a blood vessel, the distal end 112 of the catheter 100 is steered to a clot location within the blood vessel. As shown in FIG. 2A, negative pressure creates a flow towards the proximal end of the catheter 100, and the one-way valve 114 opens to aspirate clot and blood adjacent to the distal end 112. When positive pressure is applied and the flow is reversed towards the distal end 112, the one-way valve 114 closes and non-clotted blood is forced through and is filtered by the micro-valves 116. In this manner, the non-clotted blood is returned to the blood stream and the aspirated clot is maintained in the catheter 100. In this way, clot may be aspirated without removing a substantial amount of non-clotted blood and without a second blood vessel access point.

FIG. 3A shows embodiments of the end valve open, and FIG. 3B shows embodiments of the end valve closed. The one-way valve 114 at the distal end 114 of the distal portion 110 of the catheter 100 may take on a variety of shapes and design now known or later developed. Examples of designs 114a, 114b are shown in both an open and a closed position. These designs are merely exemplary of possible one-way valves that may be used. Other designs of one-way valves are also possible.

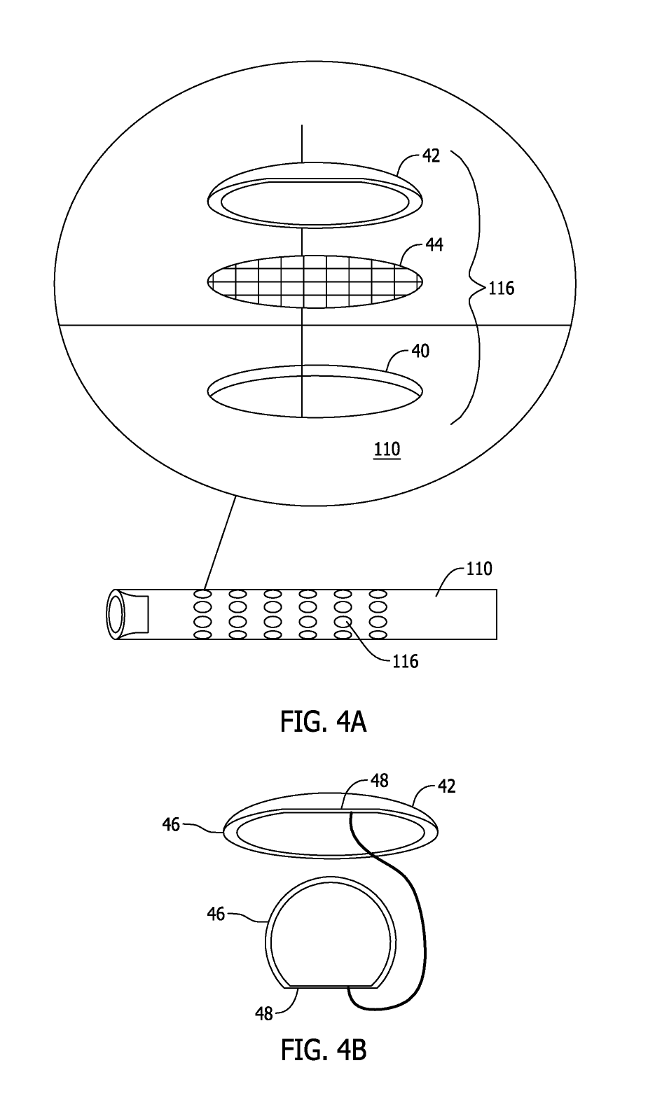

FIG. 4A shows an enlarged view of a single micro-valve, and FIG. 4B shows a perimeter of the microvalve shown in FIG. 4A. In some embodiments, the microvalves 116 may be comprises of a hole 40 within the distal portion 110 of the catheter 100, a filtration portion 44 and valve portion 42. The valve portion 42 comprises a perimeter 46 that is mostly sealed to the hole 40. The perimeter comprises a non-sealed side 48 through which filtered, non-clotted blood may flow under positive pressure. The perimeter 46 and non-sealed side 48 may be shaped as shown in FIG. 4B, or the non-sealed side 48 may continue in an arced manner. Other shapes are also possible.

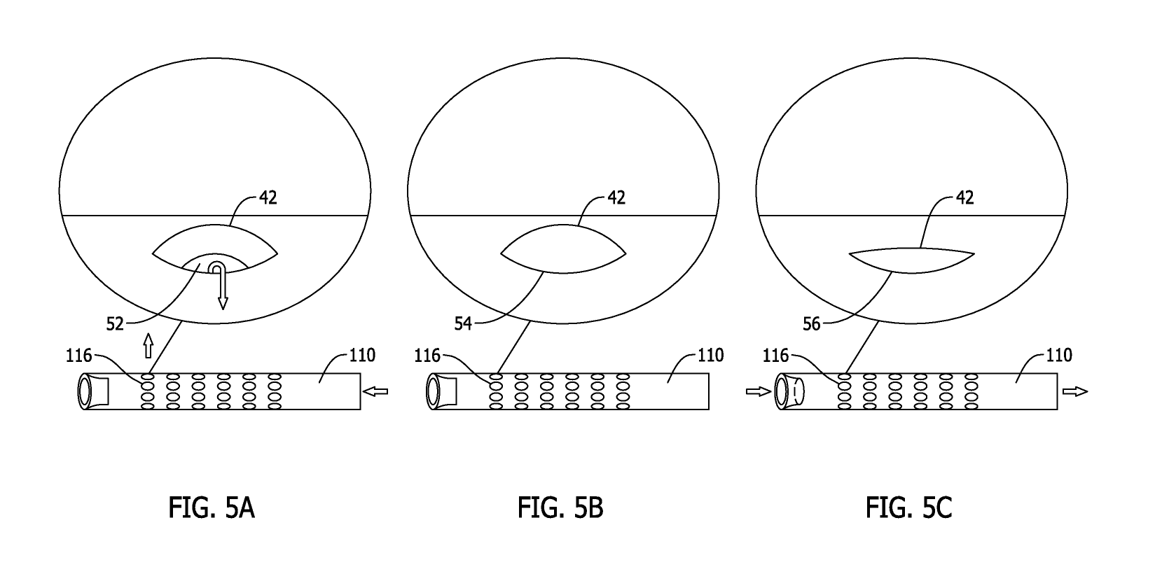

FIG. 5A, FIG. 5B, and FIG. 5C show views of the distal portion of the catheter with enlarged views of micro-valve function with various flow directions. As shown in FIG. 5A when positive pressure creating flow towards the distal portion 110 of the catheter 100 is provided, the pressure pushes non-clotted blood through an opening 52 of the valve 42 of the micro-filters 116. Specifically, referring also to FIG. 4A, the opening 52 is created by unsealed portion 48 of the perimeter 46 lifting away from the distal portion 110 of the catheter 100. Under neutral conditions shown in FIG. 5B, the valve 42 remains sealed condition 54. In FIG. 5C, under negative pressure, the valve 42 continues to be in a sealed condition 56.

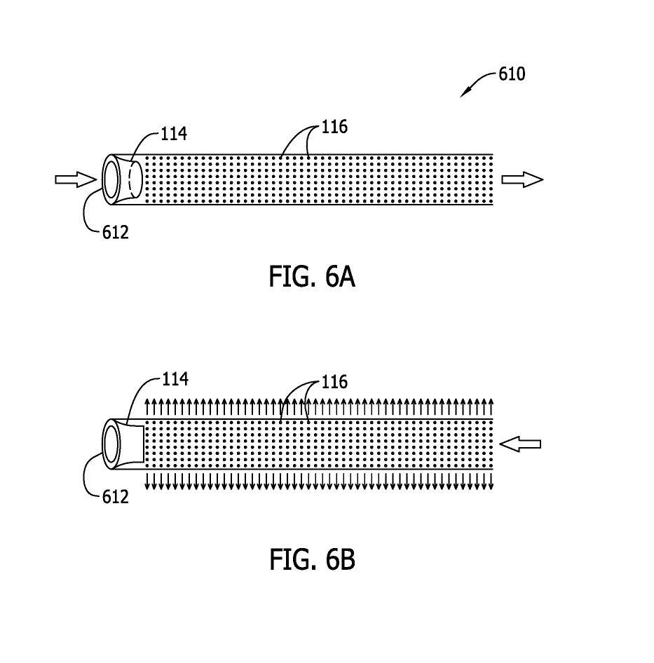

FIG. 6A shows an aspiration filter with micropores with the flow direction with aspiration, and FIG. 6B shows the flow direction with flushing with filtered fluid exiting the catheter through the micropores, according to an exemplary embodiment. In FIG. 6A, a distal portion 610 of a catheter includes a distal end 612 with the one-way valve 114. In this embodiment, instead of micro-valves, the distal portion 610 comprises a plurality of micropores 616. The micropores 616 may range from 150 to 300 microns in diameter and are created within the wall of the distal portion 610 of the catheter. The micropores 616 allow only non-clotted blood to flow outward and back into the patient while thicker, clotted blood will remain in the main lumen of the catheter. FIG. 6A shows the flow direction during an aspiration phase where the one-way valve 114 is open allowing blood and clot to continue through the catheter to a syringe, for example.

FIG. 6B shows the flow towards the distal end where the flushing flow closes the one-way valve 114 and forces non-clotted blood through the micropores 616 back into the patient.

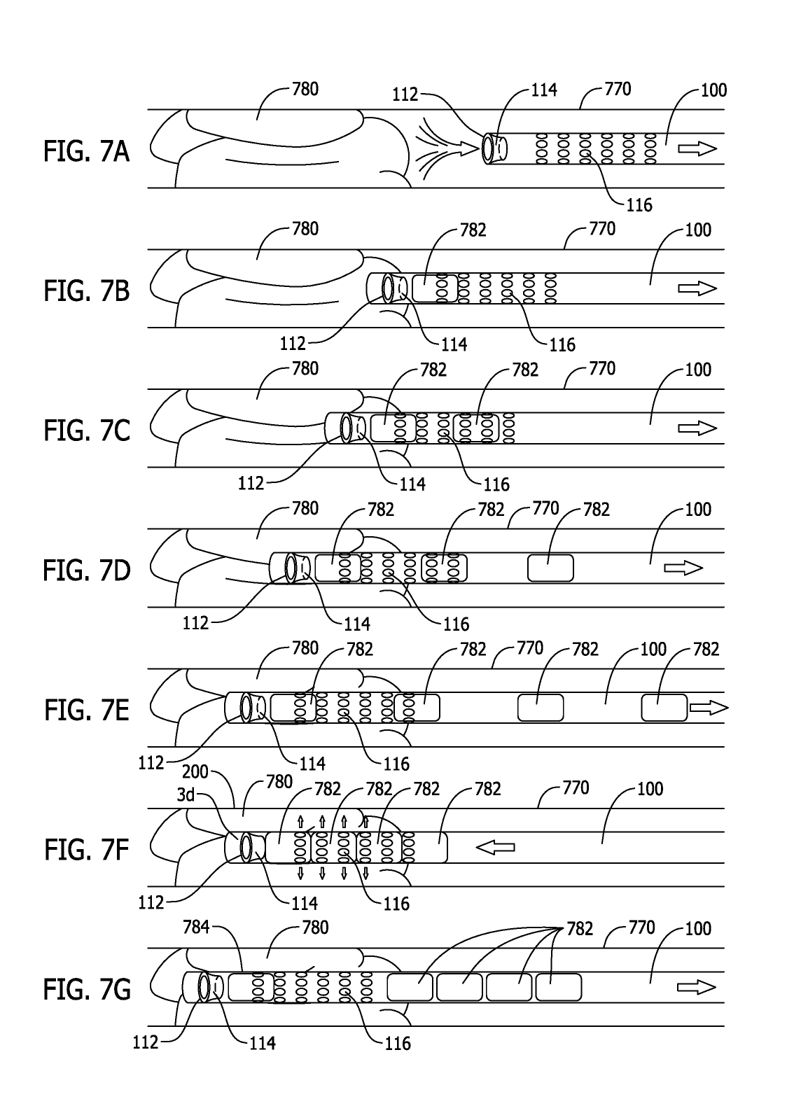

FIGS. 7A-7B show a method of using a blood clot aspiration catheter. FIG. 7A shows an aspiration catheter pre-engaged with suction and proximal flow. In FIG. 7A, the catheter 100 steered within a blood vessel 770 so that a distal end 112 is adjacent to a clot 780. Negative pressure is applied to open the one-way valve 114 to begin aspirating blood and clot 780. FIG. 7B shows the catheter engaged in the clot with unit clot removal. As the catheter 100 begins to aspirate clot 780, clot units 782 are pulled into the catheter. FIG. 7C, FIG. 7D, and FIG. 7E show further clot removal, where several clot units 782 are aspirated into the catheter 100 proceeding proximally up the catheter. During this process, non-clotted blood is also aspirated into the catheter 100.

Accordingly, FIG. 7F shows reversal of flow with expulsion of the non-clotted blood and compaction of the clot units. When positive pressure is applied, the clot units 782 are pushed back distally but are stopped from exiting the distal end 112 of the catheter via the one-way valve 114. Meanwhile, the non-clotted blood can pass through the micro-valves 116 and back into the patient. FIG. 7G shows reversal of flow and clot aspiration through the distal end valve. In FIG. 7G, negative pressure is again applied to the catheter 100 removing the clot units 782 proximally through the catheter 100 and aspirating further clot units 784 from the clot 780. These steps can be repeated until the clot is removed.

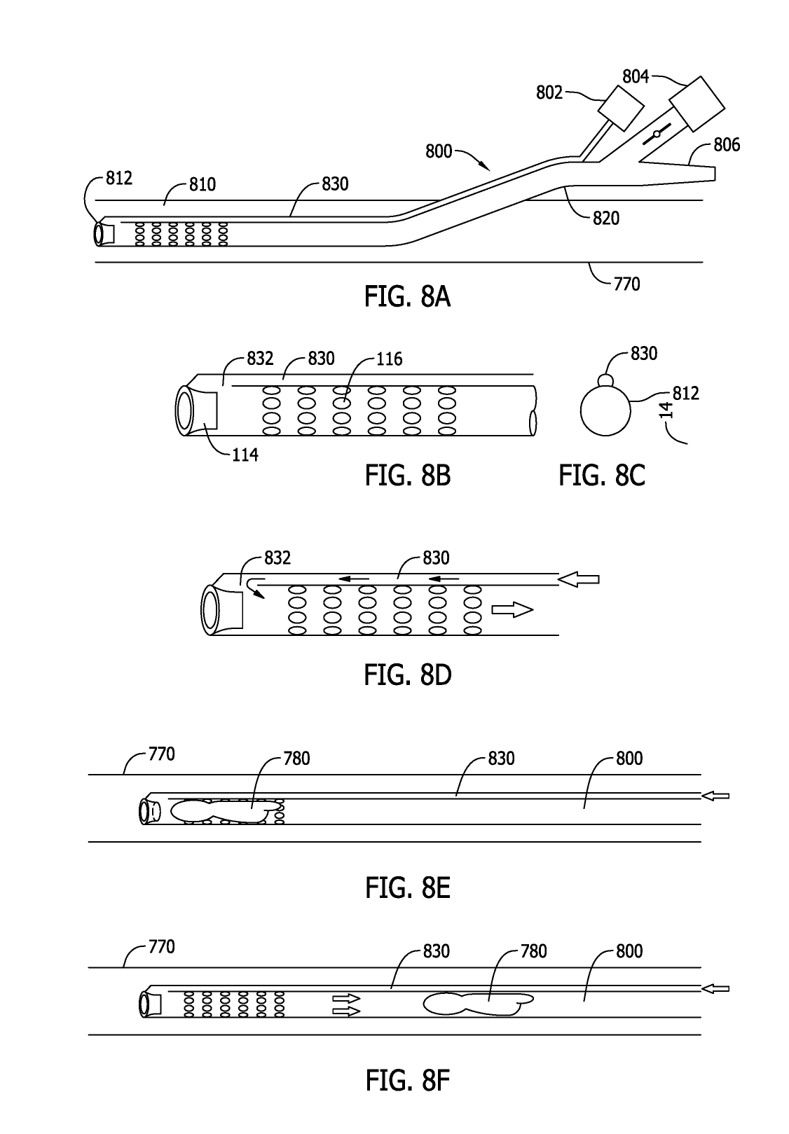

FIG. 8A shows a blood clot aspiration catheter with a small parallel catheter, according to an exemplary embodiment, FIG. 8B shows an enlarged distal portion of the catheter of FIG. 8A, and FIG. 8C shows an end view of the catheter of FIG. 8A. In this embodiment, fluid from outside the patient may be introduced to the catheter to remove clots from the catheter after the non-clotted blood has been filtered back into the patient. The catheter 800 comprises a distal portion 810, a proximal portion 820, and a small, parallel lumen 830 running the length of the catheter 800. The proximal portion 820 is configured to be outside of the patient and includes a first hub 802 for fluid insertion into the lumen 830. A second hub 804 is provided to attach a syringe or other device for providing positive and negative pressure the catheter 800. A proximal end 806 may be provided to connect to a containment device for retaining clots aspirated by the catheter.

The distal portion 810 includes a distal end 812 with a one-way valve 114 and micro-valves 116 similar to the catheter 100. The distal end 812 further comprises an open intersection 832 allowing flow from the lumen 830 to enter the catheter 800 from the distal end 812. This is shown in FIG. 8D where fluid pressure is forced through the small parallel lumen 830 and through the intersection 832 into the catheter 800. This allows an aspirated clot within the catheter 800 to be cleared. FIG. 8E shows an aspirated clot 780 within the catheter 800. As fluid flows from the lumen 830 and into the catheter 800 from the distal end, the clot 780 moves proximally through the catheter 800, for example as shown in FIG. 8F.

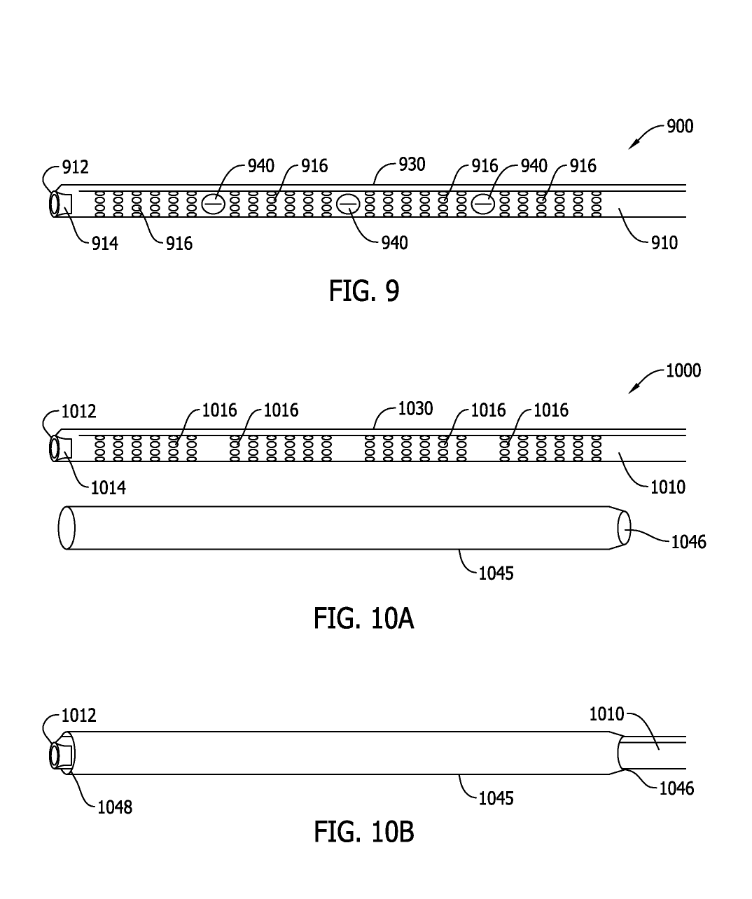

FIG. 9 shows a blood clot aspiration catheter with multiple aspiration valves along the catheter length, according to one exemplary embodiment. In some instances, a clot in a blood vessel may extend a considerable length along the blood vessel. Accordingly, there may be a catheter 900 with a distal portion 910 that comprises a distal one-way valve 914 at a distal end 912, and one or more side-wall one-way valves 940 along the length of the distal portion 910. Several micro-valves 916 are provided between the one-way valves 914, 940. The one-way valves 914, 940 and the micro-valves 916 operate similarly to valve 114 and micro-valves 116, so an explanation of these is omitted. The catheter 900 may also have a parallel lumen 930 similar to lumen 830 described above. The number of one-way valves 940 and micro-valves 116 may vary depending on vessel size, the clot burden, the age of the clot, or other factors known to those skilled in the art.

FIG. 10A shows a catheter with multiple microvalves along the catheter length and an accompanying catheter cover, according to one exemplary embodiment, and FIG. 10B shows the catheter of FIG. 10A with the cover attached. Here, a catheter 1000 may comprise a distal portion 1010 with a one-way valve 1014 at distal end 1012. The catheter 1000 also has several micro-valves 1016 to filter non-clotted blood back into the blood stream. The extra micro-valves 1016 may help to facilitate quicker filtration of the non-clotted blood. In some instances, only a portion of the distal portion 1010 may be in the blood vessel, resulting in some of the microvalves 1016 being located outside of the vessel or outside of the patient. To ensure that the filtered blood returns to the blood vessel, a sleeve or cover 1045 is provided. The cover 1045 is sealed at its proximal end 1046 to the catheter 1000 at a location proximal to the micro-valves 1016. The cover 1045 is open at the distal end 1048 allowing filtered, non-clotted blood to flow out the distal end 1048, ensuring that the non-clotted blood is returned to the blood vessel.

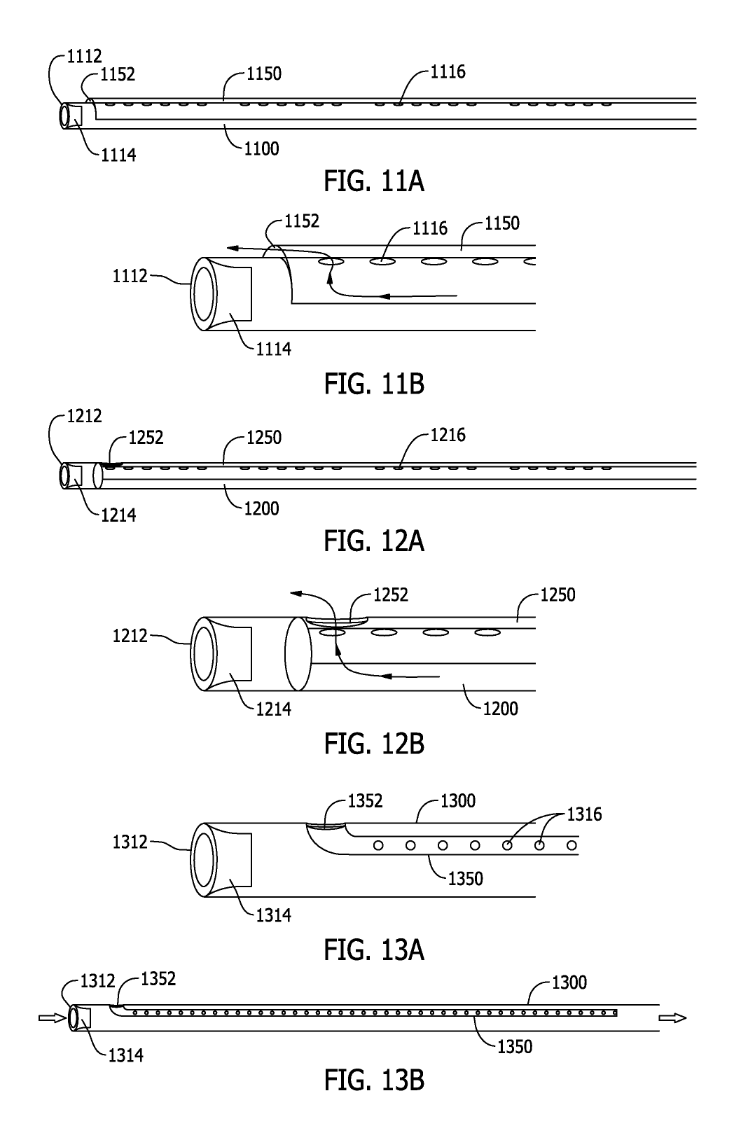

Other methods of ensuring that the filtered, non-clotted blood returns to the blood vessel are also contemplated. FIG. 11A shows an aspiration catheter with a central aspiration lumen with numerous outflow valves into a return catheter channel which continues to an outflow lumen, according to an exemplary embodiment, and FIG. 11B shows an enlarged view of the distal end of the catheter shown in FIG. 11A. In FIG. 11A, a catheter 1100 comprises several microvalves 1116 in a row on one side of the catheter. A cover 1150 is formed over the catheter 1100 and sealed on both sides of the row of microvalves 1116 creating a channel having an open distal end 1152. This allows flow to go through the micro-valves 1116 and out the distal end 1152 of the cover 1150, filtering blood aspirated through the distal end 1112 of the catheter 1100 via the one-way valve 1114.

FIG. 12A shows an aspiration catheter with a central aspiration lumen with numerous outflow valves into a return catheter channel which continues to the outflow lumen, according to an exemplary embodiment, and FIG. 12B shows an enlarged view of the distal end of the catheter shown in FIG. 12A. In FIG. 12A, a catheter 1200 comprises several microvalves 1216 in a row on one side of the catheter. The catheter 1200 is formed inside an outer cover 1250 creating a channel between the cover 1250 and catheter 1200. A distal aperture 1252 is provided at a distal end of the catheter 1200. The one-way valve 1214 is formed at a distal end of the cover 1250. This allows flow to go through the micro-valves 1216 and out the aperture 1252 of the cover 1250, filtering blood aspirated through the one-way valve 1114.

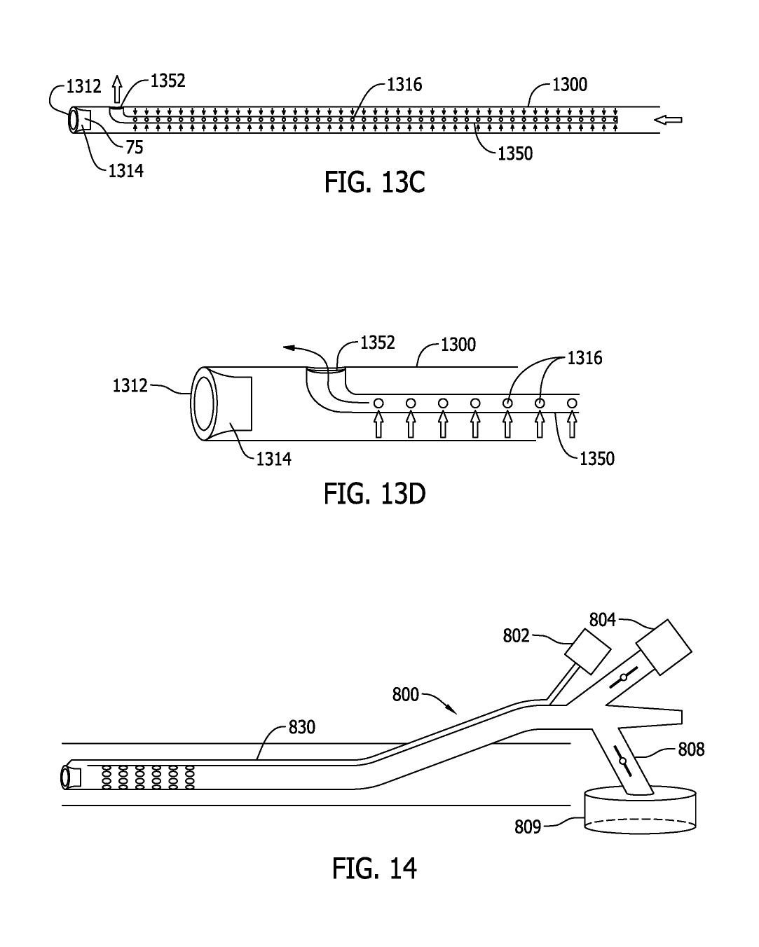

FIG. 13A shows an enlarged view of a distal end of an aspiration catheter with an internal filtration catheter. In FIG. 13A, a catheter 1300 comprises an internal filtering catheter 1350 that comprises micro-valves 1316. A distal end of the filtering catheter 1350 is attached to a wall of the catheter 1300 and includes a port 1352.

FIG. 13B shows a distal portion of the aspiration catheter of FIG. 13A during an aspiration phase. When negative pressure is applied causing flow towards a proximal end, the one-way valve 1314 at the distal end 1312 is opened to aspirate blood and clot. FIG. 13C shows the aspiration catheter of FIG. 13A during a flushing phase, and FIG. 13D shows an enlarged view of the distal end of the aspiration catheter of FIG. 13A during the flushing phase. When positive pressure is applied causing flow towards the distal end 1312, the one-way valve 1314 is closed and non-clotted blood is filtered through the micro-valves 1316 of the internal filtration catheter 1350. The non-clotted blood is the pushed out the opening 1352 at the distal end of the internal catheter 1350.

FIG. 14 shows an aspiration catheter that employs a port with a valve that directs clot to a holding container, according to an exemplary embodiment. In FIG. 14, a catheter 800 is connected to a containment bin 809. A port or valve 808 may be operated to allow an aspirated clot to move into the containment bin 809. Each of the hubs 802 and 804 may also include ports or valves to direct flow within the catheter as needed.

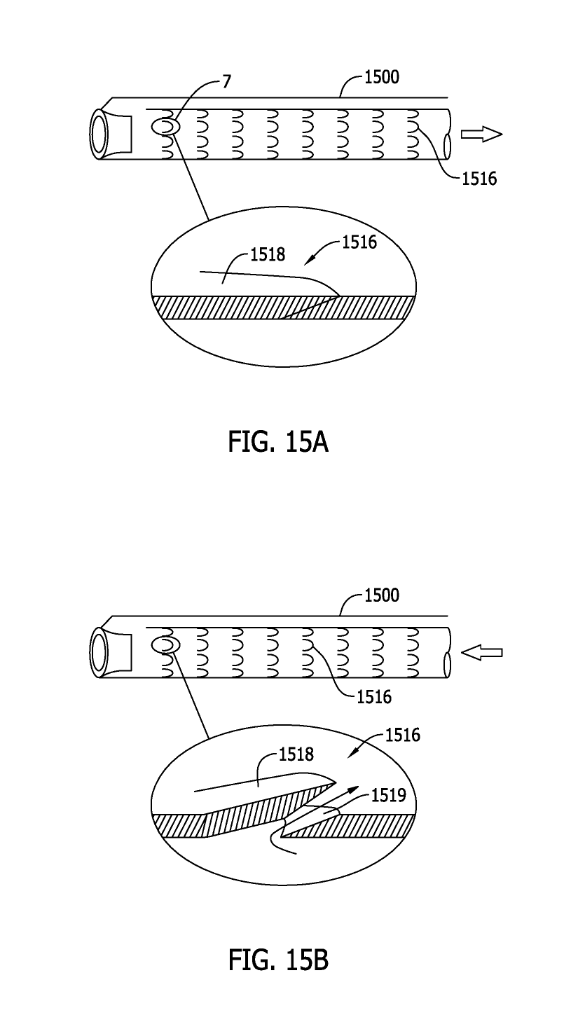

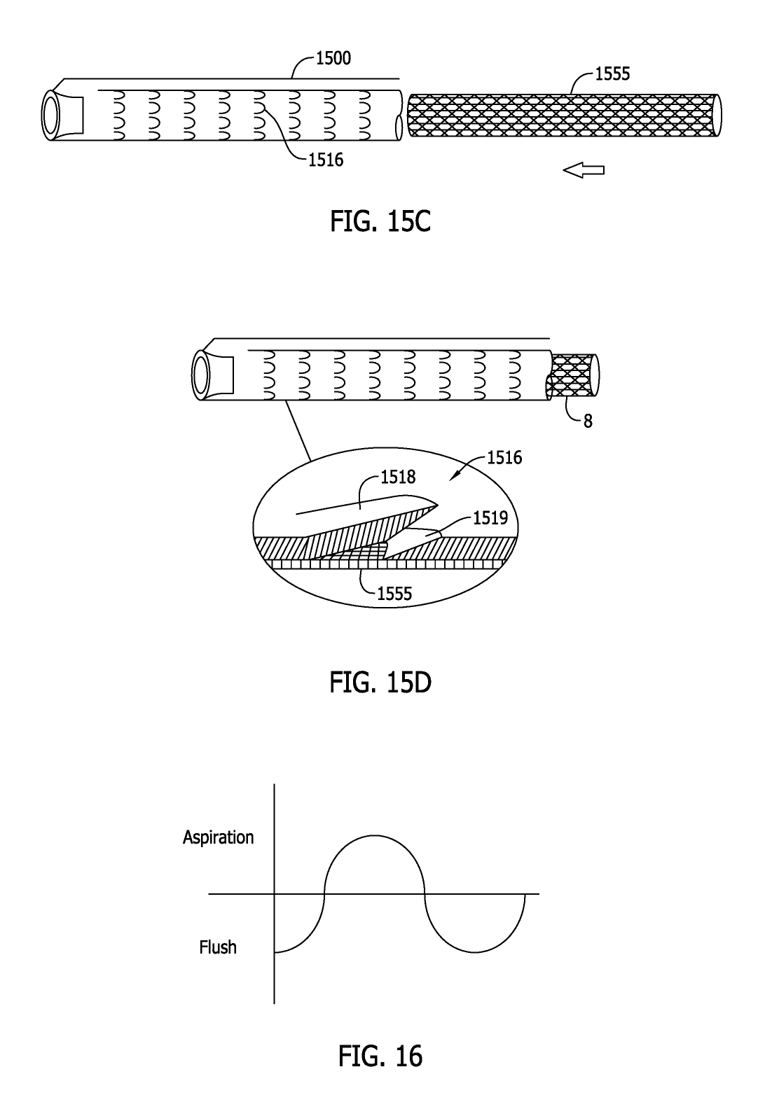

Other modifications of the above catheters are also possible. For example, each of the above-described catheters may include the micro-valves or micro-pores described above. Further, slits within the catheter may be used to filter the non-clotted blood out of the catheter. FIG. 15A shows a distal end and an enlarged section view of an aspiration catheter with slits creating the micro-valves, according to one exemplary embodiment, FIG. 15B is an enlarged view of the slit in an open state. In FIG. 15A, a catheter 1500 comprises several slits 1516 formed in a wall thereof. The slits 1516 are formed at an oblique angle creating a flap 1518. Because of the oblique flap 1518, the slit 1516 remains closed during neutral or negative pressure, and opens on positive pressure. For example, in FIG. 15B when positive pressure is applied, the flap 1518 is forced upwards creating an opening 1519 allowing the non-clotted blood to escape out of the catheter 1500.

FIG. 15C shows an internal filter to be inserted within the aspiration catheter of FIG. 15A, and FIG. 15D shows an enlarged view of the slit in an open state with the internal filter. In some embodiments, to add filtration to the slits 1516, an internal filter sleeve 1555 is provided that is concentric with the catheter 1500. When positive pressure is applied to the catheter 1500, the flap 1518 of the slit 1516 rises creating opening 1519. The blood is filtered through the sleeve 1555 and out of the opening 1519.

FIG. 16 illustrates a graph showing a possible cycle of aspiration and flushing over time, according to an exemplary embodiment. As shown in FIG. 16, the cycle may oscillate from a flush phase to an aspiration phase. Any pattern between the flushing and aspiration phases may be used.

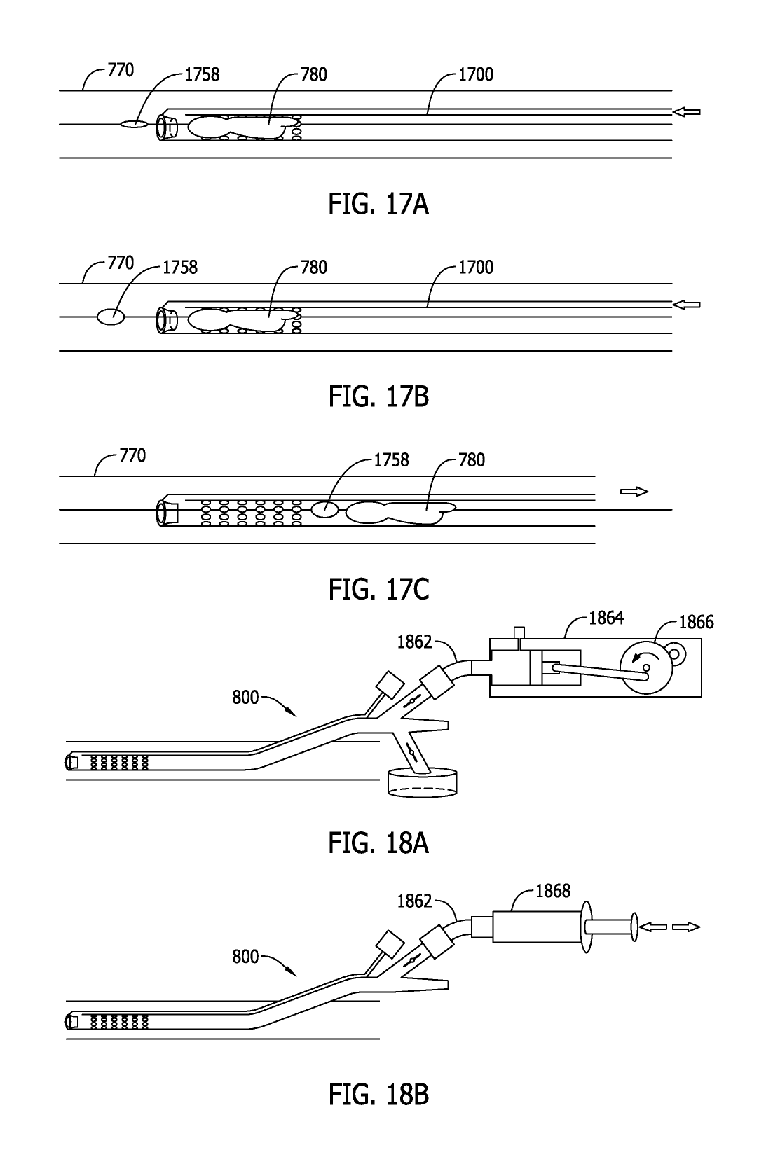

Other methods may also be used to clear clots that are aspirated into the catheter. FIG. 17A shows a deflated balloon advanced through the clot, FIG. 17B show inflation of the balloon, and FIG. 17C shows the balloon pulled pack through the catheter to remove the clot. A catheter 1700 may be used to aspirate a clot 780 within a blood vessel 770. A standard thrombectomy type balloon such as a Fogerty catheter 1758 is advanced in an ante grade fashion through the catheter 1700 in an un-inflated state. The balloon 1758 is then inflated and pulled back through the catheter 1700 dragging the clot 780 in front of it. As with the pressure means of clearance previously described the clot can be collected directly out of the back of the catheter or within the holding container.

FIG. 18A shows a motor driven cylinder creating an aspiration and a flush cycle, and FIG. 18B shows a hand operated syringe to create an aspiration and a flush cycle. To create the flush and aspiration cycles, both manual and motorized methods may be used. In FIG. 18A a catheter 800 is attached to a motorized aspiration device 1864 via a lumen 1862. The aspiration device includes a motor 1866 that drives, for example, a cylinder to create the positive and negative pressure. The motor 1866 may be driven automatically. For example, it may be controlled via a computer or other device outputting control instructions to the motor 1866. In FIG. 18B a syringe 1868 is attached to the lumen 1862 to manually create positive and negative pressure within the catheter 800. Variation in the frequency and amplitude of the cycles may vary depending on many factors such as catheter size and clot burden. As previously discussed the low pressure within the catheter draws clot inward and the high pressure filters out the non-clotted blood back into the blood vessel.

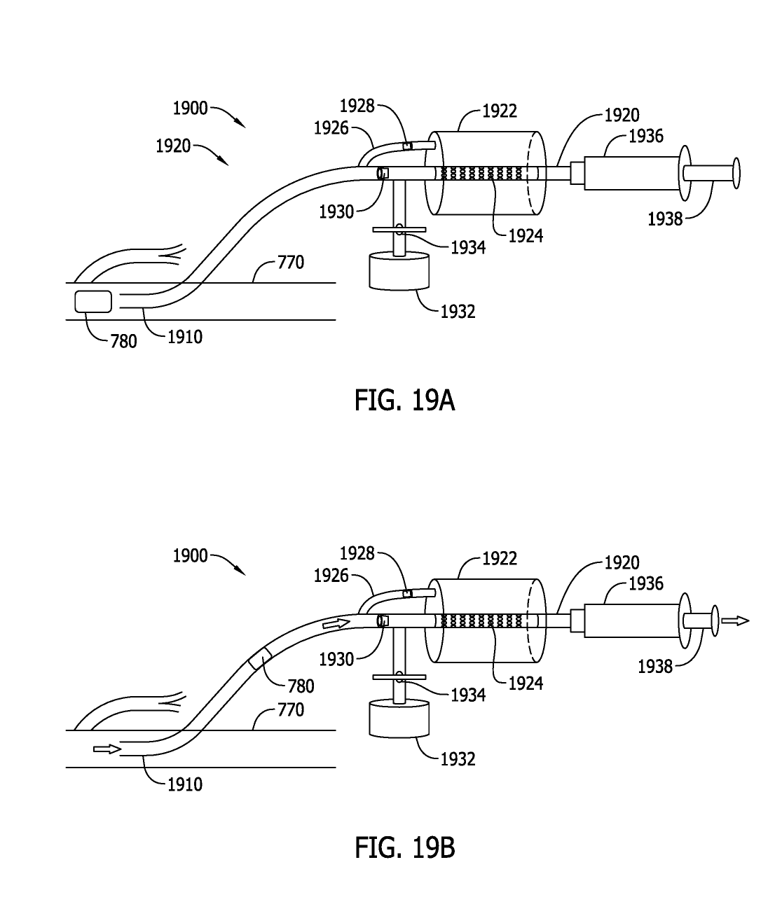

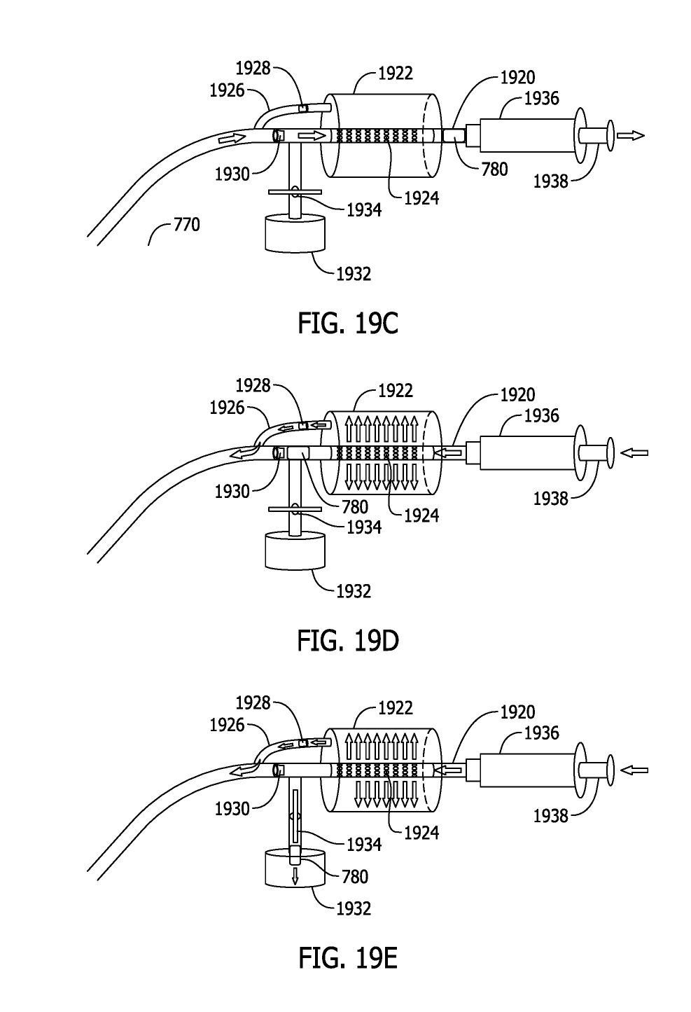

It is also contemplated that a blood clot aspiration catheter may filter non-clotted blood outside of the patient while still returning the non-clotted blood via a single venous access. FIG. 19A shows a blood clot aspiration catheter facilitating blood filtration outside of a patient, according to an exemplary embodiment. A catheter 1900 includes a distal portion 1910 that is inserted into a blood vessel 770 of a patient near a clot 780. A proximal portion 1920 comprises a fluid collector unit 1922 that houses micro-valves 1924. A one-way valve 1930 is distally located from the fluid collector unit 1930 to control the flow into and out of the collector unit 1930. A filtered blood return lumen 1926 is provided with a valve 1928 preventing backflow into the unit 1922. A collection bin 1932 is also provided proximally from the one-way valve 1930 and distally from the collector unit 1922. Access to the collection bin 1932 is controlled via port 1934.

FIG. 19B shows a clot aspirated into the filter of FIG. 19A. When negative pressure is applied to the catheter 1900 by pulling the plunger 1938 of the syringe 1936, a clot 780 is aspirated into the catheter 1900. FIG. 19C shows a proximal portion of the catheter of FIG. 19A with the clot 780 aspirated into the proximal portion 1920. In FIG. 19D, positive pressure is applied into the proximal portion 1920 of the catheter 1900, closing the one-way valve 1930 and filtering non-clotted blood through the micro-valves 1924 and returning blood to the vessel via the return lumen 1926. FIG. 19E shows clot collection in the bin 1932 when the port 1934 is opened, and continued filtered blood return using the return lumen 1926.

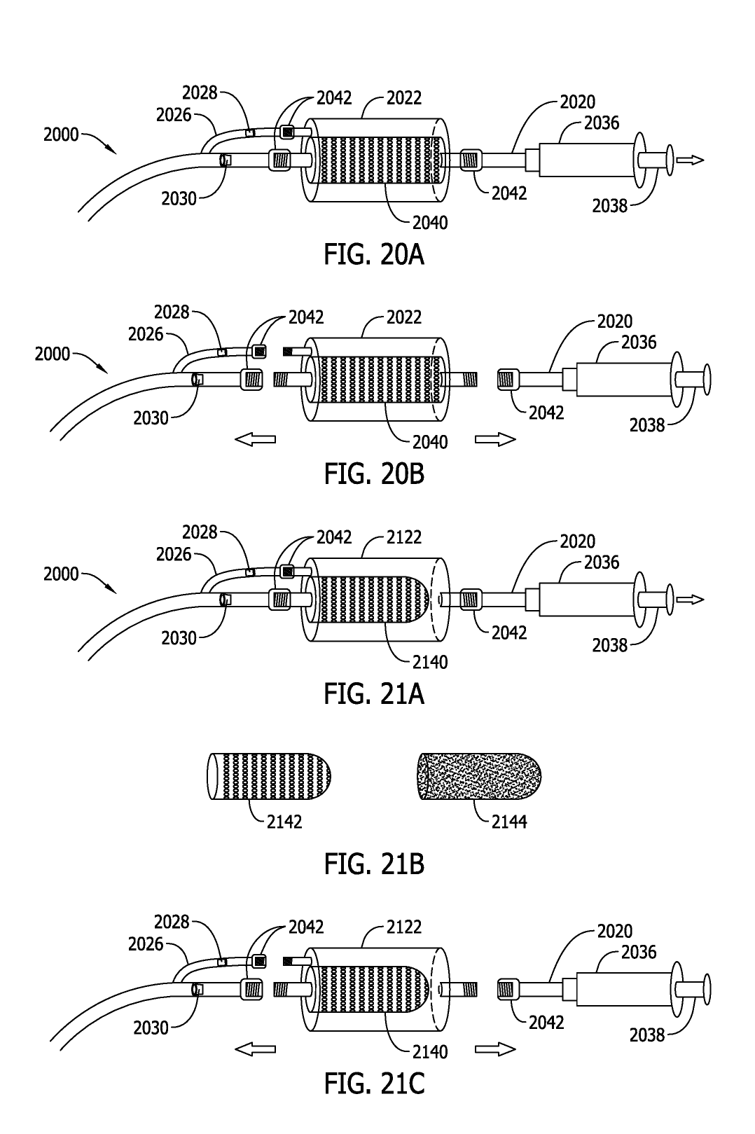

FIG. 20A shows a removable aspiration filter for an aspiration catheter, according to one exemplary embodiment, and FIG. 20B shows the filter in a removed state. In FIG. 20A, instead of a collection bin, a collector unit 2022 is provided that is removable via couplings 2040. This allows the operator to accumulate clot in the filter 2040 of the unit 2022. Once full, the collector unit 2022 is replaced with a clean collector unit 2022. Specifically, blood and clot are aspirated through the one-way valve 2030 into the collector unit 2022 and syringe 2036 when the plunger 2038 is retracted. When the plunger 2038 is pushed in, non-clotted blood is filtered through the filter 2040 and returned to the vessel via the return lumen 2026 of the catheter 2000 as regulated by valve 2028. The clots remained trapped within the filter 2040 until the collector unit 2022 is removed and replaced.

FIG. 21A shows a removable aspiration filter with a blind end filtration unit, FIG. 21 B shows a blind end filtration unit with microvalves and a mesh filtration system, and FIG. 21C shows the filter in a removed state. In FIG. 21A, an alternate collection unit 2122 may be used with a blind filter 2140. In this case, the filter 2140 filters blood during the aspiration phase as the negative pressure pulls the blood through the filter 2140. As shown in FIG. 21B, the filter may be comprised of small valves or holes as shown in filter 2142, or may be comprised of a mesh with pores of approximately 170 microns to filter blood, as shown in filter 2144. When the collector unit 2122 fills with clot, the unit 2122 is removed and replaced.

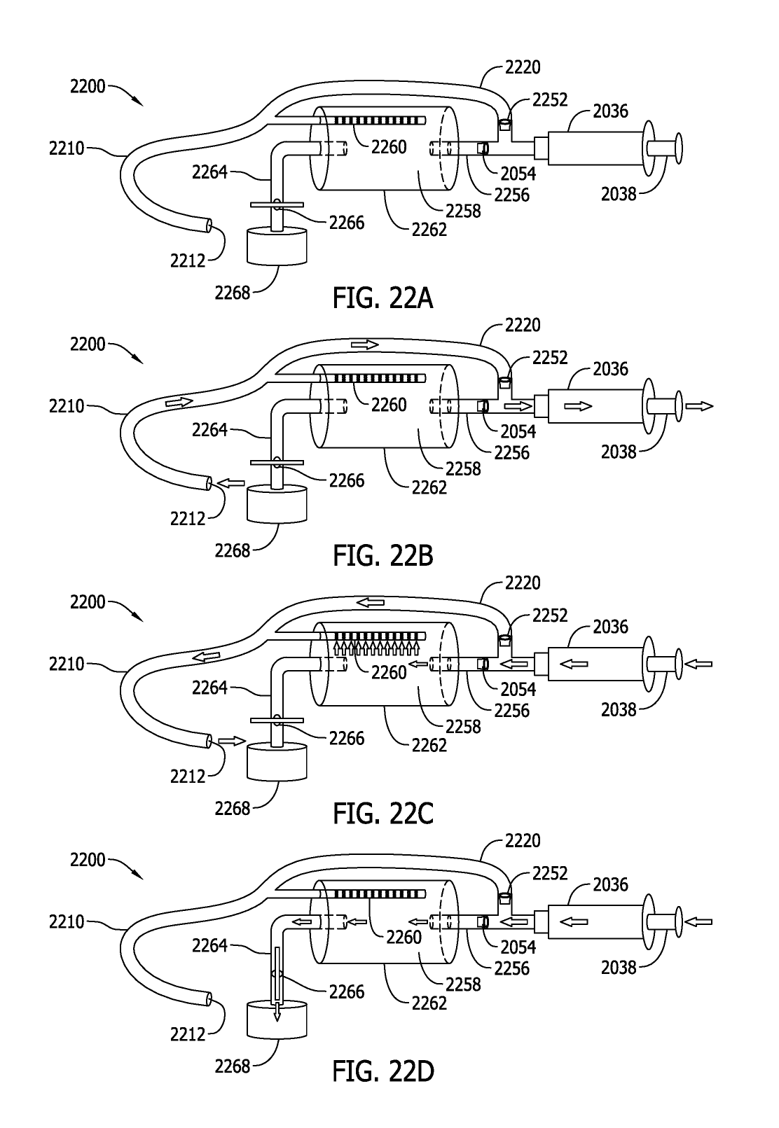

FIG. 22A shows another embodiment of a blood clot aspiration catheter. In this embodiment, catheter 2000 comprises a large clot reservoir 2262 which houses a blood filtration system 2260 that returns non-clotted, filtered blood back into the patient through the distal portion 2210 of the catheter 2200. FIG. 22B shows an aspiration phase of the catheter 2200. In the aspiration phase, flow is drawn through inlet lumen 2220, through one-way valve 2252, and into the syringe 2036. When positive pressure is applied in FIG. 22C showing a flush state of the catheter 2200, flow reverses out of the syringe 2036, through one-way valve 2254, and into the clot reservoir 2262. When the clot reservoir is full, the flush state filters the blood through the filter 2260 and returns the blood to the vessel of the patient. In FIG. 22D, clots are disposed when port 2266 is opened allowing the clot to travel through clot lumen 2263 into bin 2268.

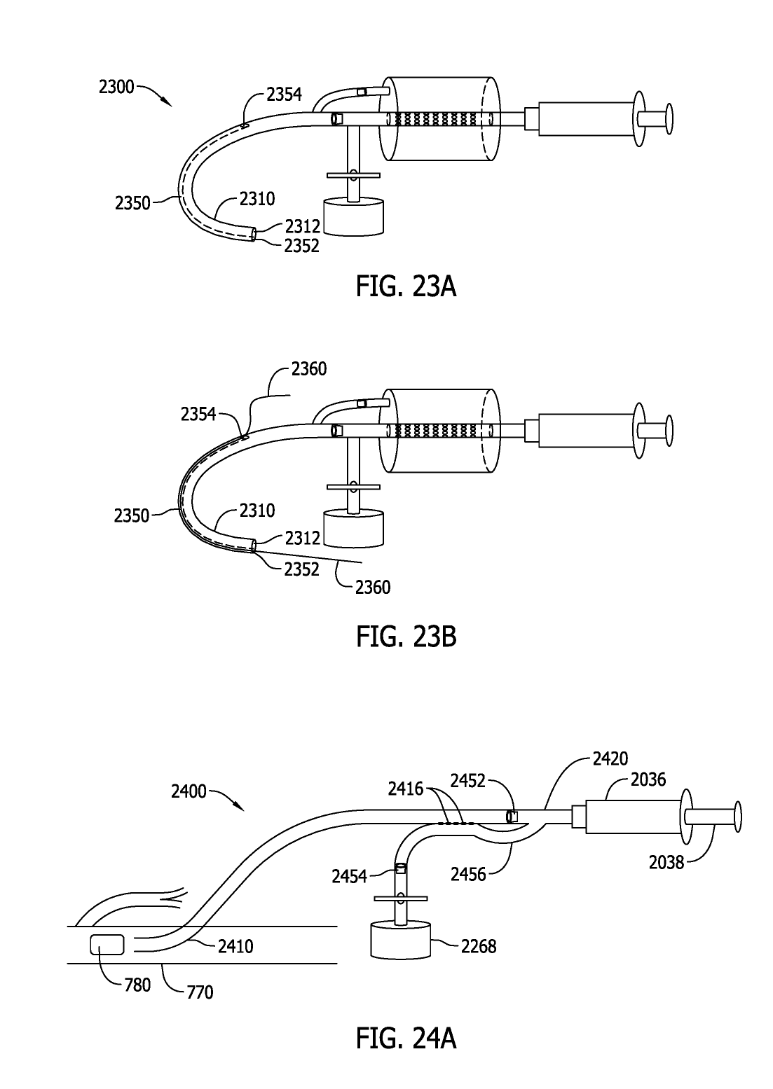

FIG. 23A shows another embodiment of a blood clot aspiration catheter with a parallel guidewire lumen, and FIG. 23B shows a guidewire inserted into the lumen. In FIG. 23A, a catheter 2300 is provided with a parallel lumen 2350 having a proximal inlet 2354 and a distal outlet 2352 at a distal end 2312 of the catheter 2300. A guidewire 2360 may be inserted into the lumen 2350 to help maintain vessel access, steer the catheter 2300, and protect the walls of the vessel.

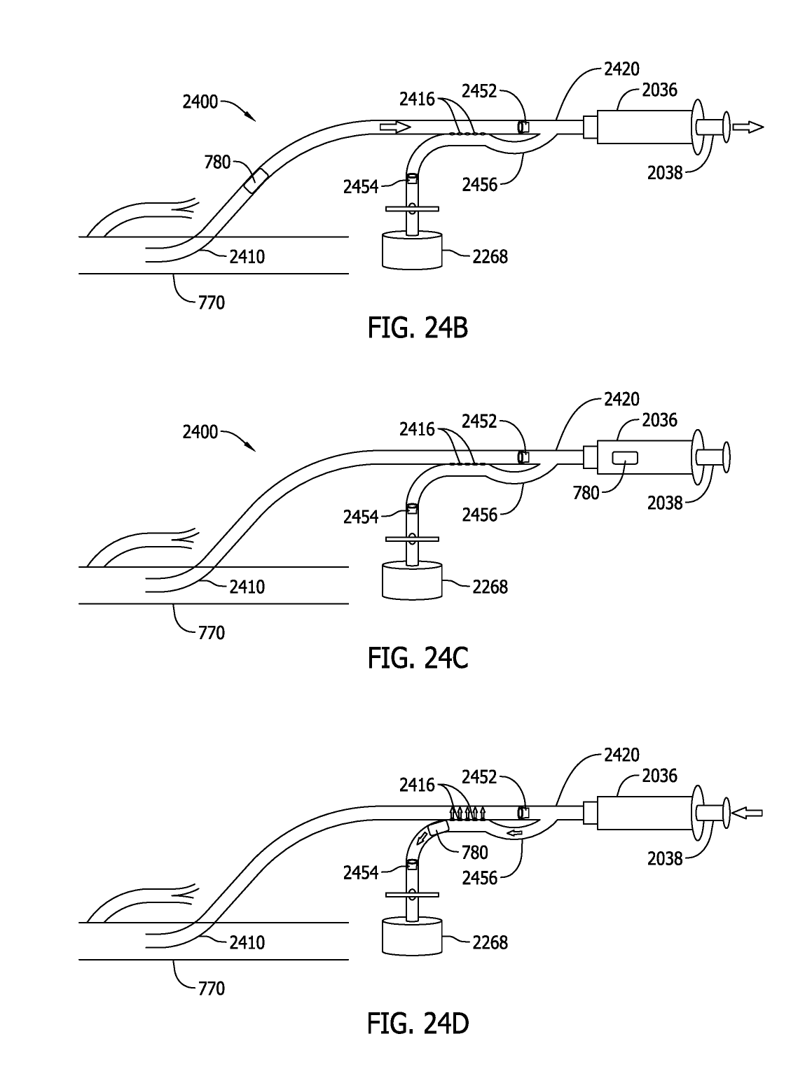

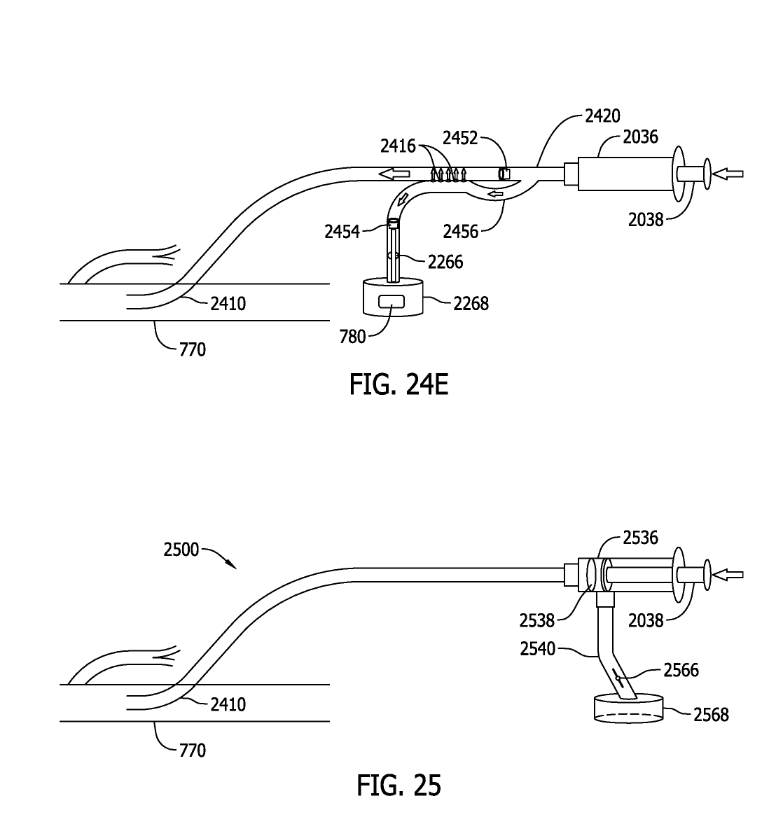

FIG. 24A shows another embodiment of a blood clot aspiration catheter with a conjoined aspiration lumen and filtration lumen attached with a connection having micro filters. In FIG. 24A, a catheter 2400 is provided having a distal portion 2410 and a proximal portion 2420.

The distal portion 2410 is steered within a blood vessel 770 to a clot 780. The proximal portion comprises a one-way valve 2452 and a filter lumen 2456. In an aspiration phase as shown in FIG. 24B, a syringe 2036 is used to pull blood and the clot 780 into the syringe 2036, shown in FIG. 24C. FIG. 24D shows forward fluid pressure from the syringe 2036 moving the clot 780 into the filtration lumen 2456. Non-clotted blood is forced through micro-valves 2416. The forward pressure closes the one-way valve 2452 diverting the blood into the filtration lumen 2456. FIG. 24E shows opening of a flow valve 2454 and passage of clot 780 into a holding container 2268.

FIG. 25 shows another embodiment of a blood clot aspiration catheter with a valve and filter combined and residing within a syringe. In FIG. 25, a filter/valve combination 2538 is disposed with a syringe 2536. It is noted that while the filter/valve combination 2538 is shown in the syringe 2536, the filter valve combination could be outsides of the syringe in series with the catheter 2500. Located within the syringe 2536 proximal to the filter valve 2538 there is a side tube 2540 with a flow switch 2566 that drains into the collecting container 2568.

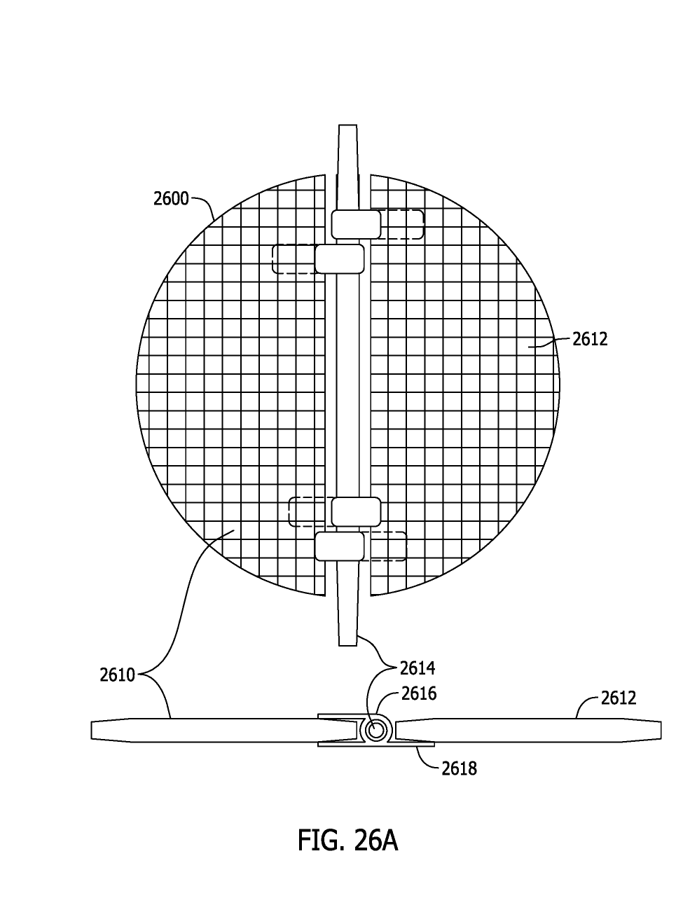

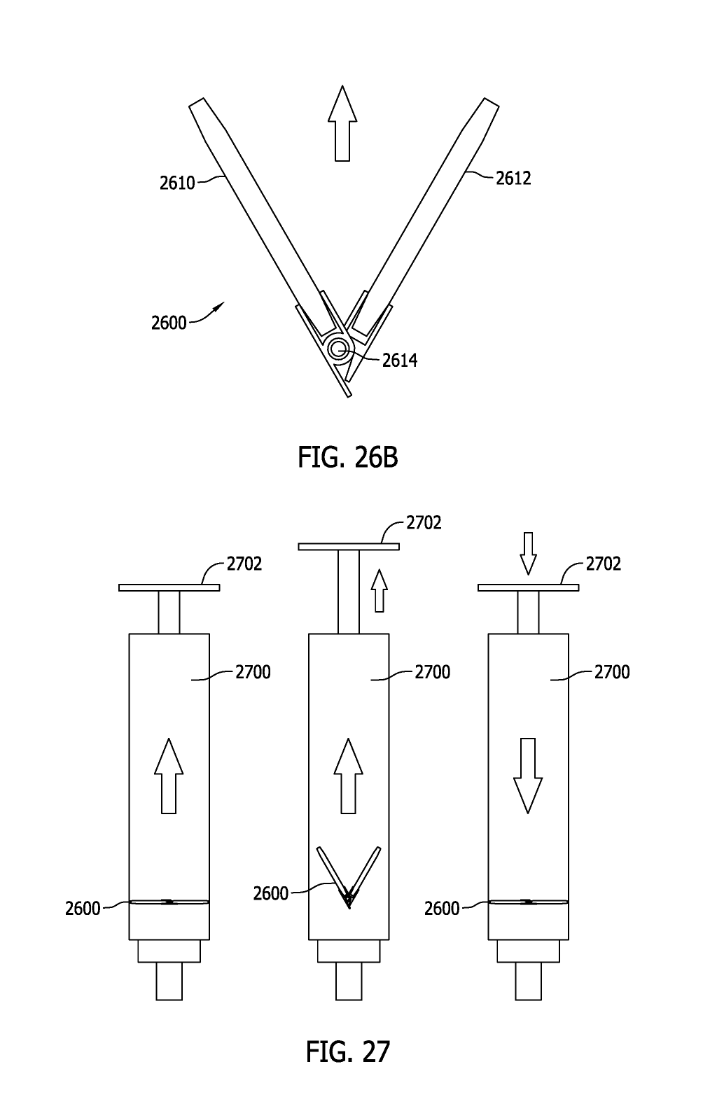

FIG. 26A shows a combination filter/valve with a central axis rod holding valve components through attached hinges that have valve stops extending from the hinges, according to one exemplary embodiment, and FIG. 26B shows the combination filter/valve in an open position. The filter valve 2600 is shown where valve hinge 2616 fits over the valve axis rod 2614 allowing for free rotation. Valve stop 2618 limits the motion of the valve 2600 keeping valve components 2610 and 2612 in line at 180 degrees in the closed position. FIG. 26B shows the filter/valve 2600 in the open position based on the direction of flow where components 2610, 2612 rotate about the center axis 2614.

FIG. 27 shows the filter/valve motion within a syringe based on neutral conditions, syringe aspiration, and flushing. In FIG. 27, when the syringe 2700 is in a neutral state, the filter/valve 2600 remains closed. When the plunger 2702 is retracted, the flow causes the filter/valve 2600 to move to the open position. And when the plunger is pushed in, the filter/valve 2600 returns to the closed position.

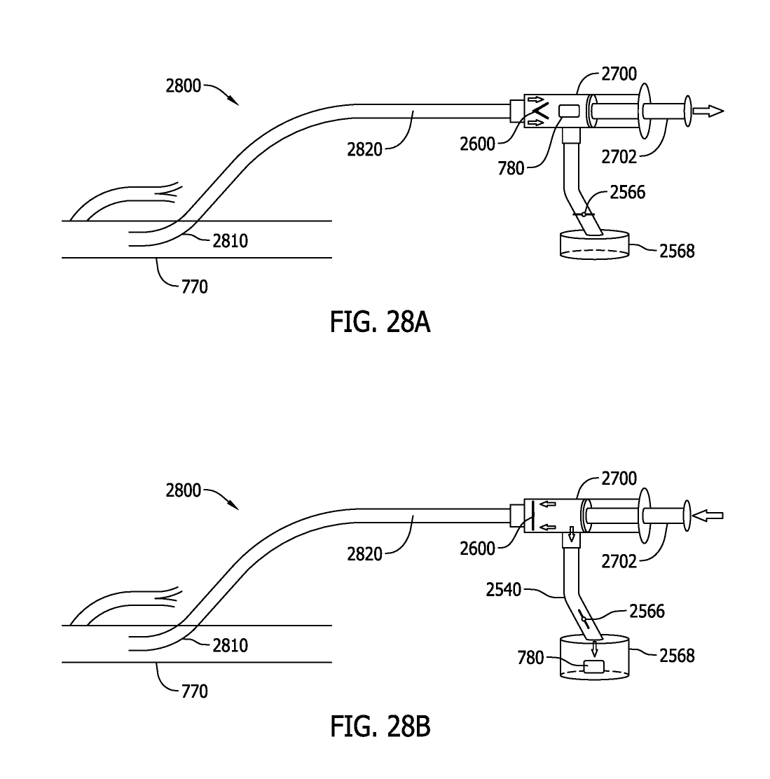

FIG. 28A shows a blood clot aspiration catheter using the combination filter valve in an aspiration phase, and FIG. 28B shows the aspiration catheter in a flush stage. In FIG. 28A, the syringe plunger 2702 is pulled back creating lower pressure in the syringe 2700 and catheter 2800 opening the filter valve 2600 allowing the clot 780 to enter the syringe 2700. With forward movement on the syringe plunger 2702 as shown in FIG. 28B, the pressure in the syringe 2700 increases and the filter valve 2600 closes filtering clotted and non-clotted blood. The clot 780 remains in the syringe 2700. By opening flow valve 2566 the clot is dropped into the collection container 2568.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A blood clot aspiration catheter comprising:
an aspiration/flushing lumen having a distal portion that is insertable into a blood vessel of a patient,
an aspiration device configured to create an aspiration phase to aspirate clot and blood into the aspiration/flushing lumen and a flushing phase to flush non-clotted blood from the aspiration/flushing lumen;
a one-way valve configured to allow clot and blood to flow through the aspiration/flushing lumen during the aspiration phase and to block flow during the flushing phase; and
filter valves configured to filter non-clotted blood to be flushed from the aspiration/flushing lumen.

2. The blood clot aspiration catheter of claim 1, wherein the one-way valve is disposed at a distal end of the catheter.

3. The blood clot aspiration catheter of claim 1, wherein the filter valves comprise micro-valves disposed on a wall of the catheter towards a distal end thereof.

4. The blood clot aspiration catheter of claim 3, wherein the micro-valves comprise a valve portion and a filter portion.

5. The blood clot aspiration catheter of claim 1, wherein the filter valves comprise micropores disposed on a wall of the catheter towards a distal end thereof, the micropores having a diameter ranging from 150 to 300 microns.

6. The blood clot aspiration catheter of claim 1, wherein the filter valves comprise slits formed at an oblique angle in a wall of the catheter towards a distal end thereof.

7. The blood clot aspiration catheter of claim 6, further comprises a filter concentric with the aspiration/flushing lumen disposed underneath the slits.

8. The blood clot aspiration catheter of claim 1, further comprising a second lumen parallel to the aspiration/flushing lumen, an intersection between the second lumen and the parallel lumen being disposed at a distal end of the catheter, and the second lumen being configured to introduce a flushing fluid into the aspiration/flushing lumen at the distal end via the intersection.

9. The blood clot aspiration catheter of claim 1, wherein the one-way valve is disposed on a distal end of the catheter and one or more side-wall one-way valves are disposed on a distal portion of the catheter proximally from the one-way valve.

10. The blood clot aspiration catheter of claim 9, wherein the filter valves are disposed between the one-way valve and the one or more side-wall one-way valves.

11. The blood clot aspiration catheter of claim 1, wherein the filter valves are disposed on a distal portion of the aspiration/flushing lumen, and the catheter further comprises a sleeve disposed over the aspiration/flushing lumen, a proximal end of the sleeve being sealed to the aspiration/flushing lumen proximally from the filter valves forming a channel between the sleeve and the aspiration/flushing lumen having an outlet at a distal end of the catheter.

12. The blood clot aspiration catheter of claim 1, wherein the filter valves are disposed in a row along the aspiration/flushing lumen, and a channel is formed over the filter valves having an outlet at a distal end of the catheter.

13. The blood clot aspiration catheter of claim 1, further comprising a filtration lumen disposed within the aspiration/flushing lumen, the filter valves being disposed on the filtration lumen, the filtration lumen being connected to the aspiration/flushing lumen towards a distal end of the aspiration/flushing lumen, and an outlet of the filtration lumen being disposed at the connection of the filtration lumen to the aspiration/flushing lumen.

14. The blood clot aspiration catheter of claim 1, further comprising a retraction balloon, wherein the retraction balloon is advance through the aspiration/flushing lumen in an uninflated state, is inflated distally from a clot aspirated within the aspiration/flushing lumen, and pushes the clot proximally through the aspiration/flushing lumen in while inflated.

15. The blood clot aspiration catheter of claim 1, wherein the aspiration device is a syringe.

16. The blood clot aspiration catheter of claim 1, wherein the aspiration device is a motorized cylinder.

17. The blood clot aspiration catheter of claim 1, further comprising a fluid collector unit disposed on a proximal portion of the catheter attached to the aspiration/flushing lumen, the filter valves being disposed within the fluid collector unit.

18. The blood clot aspiration catheter of claim 17, wherein the fluid collector unit is removable.

19. The blood clot aspiration catheter of claim 1, wherein the filter valves are disposed on the one-way valve.

20. The blood clot aspiration catheter of claim 19, wherein the filter valves are filter components rotatably attached to an axis rod to rotate and allow flow during the aspiration phase and to close and filter flow during the flushing phase.
